(19) United States
(12) Patent Application Publication
(10) Pub. No.: US 2013/0203647 A1
(43) Pub. Date: Aug. 8, 2013

(54) DELIVERY OF HYDROPHILIC PEPTIDES

(71) Applicants: Ijeoma Uchegbu, London (GB); Andreas Schatzlein, London (GB); Mariarosa Mazza, London (GB)

(72) Inventors: Ijeoma Uchegbu, London (GB); Andreas Schatzlein, London (GB); Mariarosa Mazza, London (GB)

(73) Assignee: UNIVERSITY COLLEGE LONDON, London (GB)

(21) Appl. No.: 13/809,374

(22) PCT Filed: Jul. 11, 2011
(86) PCT No.: PCT/GB2011/051288

§ 371 Date: Mar. 28, 2013

(30) Foreign Application Priority Data
Jul. 9, 2010 (GB) 1011602.8

(51) Int. Cl. A61K 47/48

(52) U.S. Cl. CPC A61K 47/48046; A61K 47/48092
USPC 514/1.3; 530/300; 530/322

(57) ABSTRACT

A composition comprises nanofibres for the delivery of a peptide across the blood brain barrier in a method of therapy of the human or animal body, wherein the nanofibres comprise a peptide conjugated to a lipophilic group. Further, a compound comprises a Dalargin or a derivative having one or more substituted, deleted or inserted aminoacyl units, and, conjugated to an aminoacyl group preferably via a side chain, a lipophilic group, optionally via a linker.

### FIELD OF THE INVENTION

The present invention relates to a new system for the delivery of hydrophilic peptides and other drugs to the brain. The system involves forming an endogenously cleavable lipophilic derivative of the hydrophilic peptide, and formulating this into nanofibres. The invention has particular utility for the oral and intra-venous delivery of hydrophilic drugs to the brain.

### BACKGROUND TO THE INVENTION

Nanofibrous systems are attracting increasing interest in the field of drug delivery and regenerative medicine^{1, 2}. We have described in a previous patent application (PCT/GB10/50355), unpublished at the priority date, compositions comprising lipophilic derivatives of hydrophilic drugs coupled with an amphiphile compound for delivery of drugs to the brain. However there remains a desire in this field to provide further improved formulations for delivery of peptides to the brain.

### SUMMARY OF THE INVENTION

The invention provides a composition comprising nanofibres for the delivery of a peptide or other drugs across the blood brain barrier in a method of therapy of the human or animal body, wherein the nanofibres comprise a peptide conjugated to a lipophilic group and wherein the peptide may be the active drug or an active drug may in turn be loaded on to the nanofibres.

Pharmaceutical compositions, methods of therapy using the above composition and methods of forming the above composition are also provided.

Significant benefits, for instance, antinociceptive effect can be achieved with the formulations of the invention. The invention is generally applicable to peptides and other drugs that are known to be largely excluded from the brain.

### DETAILED DESCRIPTION OF THE INVENTION

By lipophilic, is meant a compound having very low solubility in water (<0.1 mg/mL). By hydrophilic, is meant a compound with high water solubility (>1 mg/mL).

The invention has utility for the delivery of hydrophilic peptides to the brain. Preferably the peptide is a therapeutic agent (drug). We have shown that the peptide, delivered in accordance with the invention, is able to cross the blood brain barrier and have a therapeutic effect in the brain.

The nanofibres comprise a peptide conjugated to a lipophilic group. The lipophilic group is preferably cleavable, i.e. the nanofibres derivative may act as a pro-drug which is cleaved to the active drug in the human or animal body, preferably at the drug's target location.

Preferably, the linker is enzymatically cleavable. However, local environmental conditions within the body may alternatively promote cleavage. Low pH, in the range 1-5, and hypoxic conditions are known to promote pro-drug cleavage.

The lipophilic group renders the peptide lipophilic. Typically, the lipophilic group comprises a substituted or unsubstituted hydrocarbon group comprising at least 4 carbon atoms, preferably at least 10 or 15 carbon atoms, and comprises, for instance a C₄₋₃₀ alkyl group, C₄₋₃₀ acyl group, a C₄₋₃₀ alkenyl group, a C₄₋₃₀ alkynyl group, a C₅₋₂₀ aryl group, a multicyclic hydrophobic group with more than one C₄-C₈ ring structure such as a sterol (e.g. cholesterol), a multicyclic hydrophobic group with more than one C₄-C₈ heteroatom ring structure, a polyoxa C₁-C₄ alkylene group such as polyoxa butylene polymer, or a hydrophobic polymeric substituent such as a poly(lactic acid) group, a poly(lactide-co-glycolide) group or a poly(glycolic acid) group. The linker may be linear, branched or have cyclo groups.

Preferably, the lipophilc group is covalently attached to the hydrophilic drug. However, it need not be, and electrostatic means of association with the hydrophilic drug are also included within the scope of this invention.

Typically, the lipophilic group is attached to the hydrophilic drug by means of an acyl group. For instance, the linker may be attached via an ester or an amide linkage, with the nitrogen or oxygen atom of this linkage derived from the hydrophilic drug. For instance, the hydrophilic drug may have an amine or a hydroxyl group which is derivatised by the linker. When the hydrophilic drug is a peptide, such groups may form part of the peptide backbone or of an amino acid's side chain. For instance, the side chain hydroxyl of a tyrosine residue may be derivatised. A particularly preferred linker has the general formula —C(═O)R¹, wherein R¹ is any of the linkers outlined above and is preferably C₄₋₂₀ alkyl which may be optionally substituted with groups well known in the art, which do not detract from the linker's hydrophobicity.

A particularly preferred lipophilic group is derived from palmitic acid, i.e. a palmitoyl group. Other preferred groups are derived from caprylic, capric, lauric, myristic, stearic and arachidic acids and cholesterol.

Peptides are of tremendous clinical value for the treatment of many central nervous system (CNS) disorders, and preferably therefore the drug is a CNS active drug. Many existing peptide pharmaceuticals are rendered ineffective after oral administration or are unable to cross the blood brain barrier (BBB) on parenteral administration mainly due to their hydrophilicity, size, charge and rapid metabolic degradation in the gastrointestinal tract and blood, as detailed above. Since the invention has particular utility for delivering drugs to the brain, the hydrophilic drug is preferably a neuroactive agent.

Endogenous opioid neuropeptides, preferably neuro-penta and hexapeptides are particularly preferred drugs for use in this invention. Examples include Met⁵-Enkephalin and Leu⁵-Enkephalin.

The drug may be used to treat brain disorders such as schizophrenia, obesity, pain and sleep disorders, psychiatric diseases, neurodegenerative conditions, brain cancers and infective diseases.

Preferred drugs include neuropeptides: enkephalin, neuropeptide S, dalargin, orexin, dynorphin, detorphin I, oxytocin, vasopressin and leptin. Other preferred drugs include: cholecystokinin, gosarelin and leutenizing hormone releasing hormone. A lipophilic peptide, palmitoylated dalargin is an example of a conjugated peptide which can be used in this invention. We believe that lipophilised dalargins are novel compounds. The novel compounds are claimed in claims 15 and 16. The method of synthesising of the compounds is also claimed.

The compounds are self-assembling in aqueous dispersion and deliver to the brain.

According to a further aspect of the invention nanotubes of amphiphilic compounds are used to deliver a non-conjugated drug to the brain. The drug may be hydrophilic or lipophilic.

The peptide conjugate used in this invention is formulated into nanofibres. Nanofibres are fibres with diameters in the nanometer range, i.e. 1-1000 nm, and typically around 50 to 100 nm. The lengths of these nanofibres are in the range diameter up to around 500 μm. The amphiphilic nature of the peptide-lipophilic group allows the nanofibres to form. High axial ratio micellar aggregates can form either cylindrical or twisted nanofibres. Nanofibres can be formed by a variety of methods known in the art including probe sonication.

The nanofibres can be formulated together with a separate drug in order to deliver this drug to the brain. Examples of such drugs include lomustine, etoposide, paclitaxel, carmustine, temozolamide and doxorubicin.

The nanofibres can be formulated together with an amphiphile compound before being administered. However, in contrast to our previous invention (PCT/GB10/50355), the amphiphile does not need to be present and the nanofibres can be prepared without this being present. The amphiphile compounds useful in this invention are compounds comprising a hydrophobic moiety covalently linked to a hydrophilic moiety and typically form nanoparticles themselves. They may be selected from the following compounds: sorbitan esters, polysorbates, poly(ethylene glycol) alkyl, aryl and cholesterol ethers [e.g. phenolic and alkyl derivatives of poly(ethylene glycol)], poly(ethylene oxide)-poly(propylene oxide) block copolymers, polymer amphiphiles, phospholipids, fatty acid salts, acylated amino acids, alkyl quaternary amine salts, alkyl amine oxides, alkyl sulphonates, aryl sulphonates, C₄-C₃₀ alkyl amine salts. Preferably, the amphiphile compound is an amphiphilic carbohydrate compound.

The amphiphilic carbohydrate compound is typically selected from chitosans, dextrans, alginic acids, starches, guar gums, and their derivatives. Preferably the amphiphilic compound is a chitosan or a derivative thereof, for instance, acetylated palmitoyl quaternary ammonium glycol chitosan (GCPQA).

In a preferred embodiment of the invention, the amphiphilic carbohydrate compound is represented by the formula:

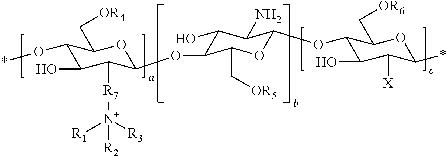

wherein a+b+c=1.000 and

a is between 0.01 and 0.990,

b is between 0.000 and 0.980, and

c is between 0.01 and 0.990;

and wherein:

X is a hydrophobic group;

R₁, R₂ and R₃ are independently selected from hydrogen or a substituted or unsubstituted alkyl group;

R₄, R₅ and R₆ are independently selected from hydrogen, a substituted or unsubstituted alkyl group, a substituted or unsubstituted ether group, or a substituted or unsubstituted alkene group;

R₇ may be present or absent and, when present, is an unsubstituted or substituted alkyl group, an unsubstituted or substituted amine group or an amide group; or a salt thereof.

In the above general formula, the a, b and c units may be arranged in any order and may be ordered, partially ordered or random. The * in the formula is used to indicate the continuing polymer chain. In preferred embodiments, the molar proportion of the c units is greater than 0.01, and more preferably is at least 0.110, more preferably is at least 0.120, more preferably is at least 0.150 or in some embodiments is at least 0.18. Generally, the molar proportion of the c unit is 0.400 or less, and more preferably is 0.350 or less.

Preferably, the molar proportion of the a unit is between 0.010 and 0.800, and more preferably between 0.050 and 0.300.

Preferably, the molar proportion of the b unit is between 0.200 and 0.850, and more preferably between 0.200 and 0.750.

As can be seen from the above formula, the b units may optionally be absent. The c units provide the first portion of the monomer units that are derivatised with a hydrophobic group, and the a units provide the second portion of the monomer units and are derivatised with a quaternary nitrogen group. When present, the b units provide the third group of monomer units in which the amine groups are derivatised in a different manner to the first or second group, or else are underivatised.

In the present invention, the hydrophobic group X is preferably selected from a substituted or unsubstituted group which is an alkyl group such as a C₄-₃₀ alkyl group, an alkenyl group such as a C₄-₃₀ alkenyl group, an alkynyl group such as a C₄-₃₀ alkynyl group, an aryl group such as a C₅-₂₀ aryl group, a multicycle hydrophobic group with more than one C₄-C₈ ring structure such as a sterol (e.g. cholesterol), a multicyclic hydrophobic group with more than one C₄-C₈ heteroatom ring structure, a polyoxa C₁-C₄ alkylene group such as polyoxa butylene polymer, or a hydrophobic polymeric substituent such as a poly(lactic acid) group, a poly(lactide-co-glycolide) group or a poly(glycolic acid) group. The X groups may be linear, branched or cyclo groups. Any of the X groups may be directly linked to the c unit (i.e. at the C2 of the monomer unit), or via a functional group such as an amine group, an acyl group, or an amide group, thereby forming linkages that may be represented as X′-ring, X′—NH—, X′—CO-ring, X′CONH-ring, where X′ is the hydrophobic group as defined above.

Preferred examples of X groups include those represented by the formulae CH₃(CH₂)ₙ—CO—NH— or CH₃(CH₂)ₙ—NH— or the alkeneoic acid CH₃ (CH₂)ₚ—CH═CH—(CH₂)_{q}—CO—NH—, where n is between 4 and 30, and more preferably between 6 and 20, and p and q may be the same or different and are between 4 and 16, and more preferably 4 and 14. A particularly preferred class of X substituents are linked to the chitosan monomer unit via an amide group, for example as represented by the formula CH₃(CH₂)ₙCO—NH—, where n is between 2 and 28. Examples of amide groups are produced by the coupling of carboxylic acids to the amine group of chitosan. Preferred examples are fatty acid derivatives CH₃(CH₂)ₙCOOH such as those based on capric acid (n=8) lauric acid (n=10), myristic acid (n=12), palmitic acid (n=14), stearic acid (n=16) or arachidic acid (n=18).

In the above formula, R₁, R₂ and R₃ are preferably independently selected from hydrogen or a substituted or unsubstituted alkyl group such as a C₁₋₁₀ alkyl group. Where R₁, R₂ and/or R₃ are alkyl groups, they may be linear or branched. Preferably, R₁, R₂ and R₃ are independently selected from hydrogen, methyl, ethyl or propyl groups.

In the above formula, R₄, R₅ and R₆ present on the C6 or the sugar units are independently selected from hydrogen, a substituted or unsubstituted alkyl group, a substituted or unsubstituted ether group, or a substituted or unsubstituted alkene group. Preferred R₄, R₅ and R₆ groups are substituted with one of more hydroxyl groups, or another non-ionic hydrophilic substituent. Examples of R₄, R₅ and R₆ groups are represented by the formulae —(CH₂)ₚ—OH, where p is between 1 and 10, and is preferably between 2 and 4, or —(CH₂)ₚ—CH_{q}(CH₂—OH)ᵣ where p is between 1 and 10 and q is between 0 and 3 and r is between 1 and 3 and the sum of q+r=3, or —(CH2)ₚ—C(CH₂—OH)ᵣ where p is between 1 and 10, and r is 3, or —(CH₂CH₂OH)ₚ, where p is between 1 and 300.

The R₇ group may be present or absent in the general formula. When absent, it provides a quaternary ammonium functional group that is directly linked to the chitosan ring of the a monomer unit. When the R₇ group is present it may be a unsubstituted or substituted alkyl group (e.g. a C₁₋₁₀ alkyl group) for example as represented by the formula —(CH₂)ₙ—, an amine group as represented by the formula —NH—(CH₂)ₙ—, or an amide group as represented by the formula —NH—CO—(CH₂)ₙ—, where n is 1 to 10 and is preferably 1 to 4. A preferred example of the R₇N⁺R₁R₂R₃ substituent is provided by coupling betaine (—OOC—CH₂—N—(CH₃)₃) to the amine substituent of the a unit providing an amide group such as in betaine, —NH—CO—CH₂—N⁺R₁R₂R₃.

As indicated, some of the substituents described herein may be either unsubstituted or substituted with one or more additional substituent's as is well known to those skilled in the art. Examples of common substituent's include halo; hydroxyl; ether (e.g., C₁₋₇ alkoxy); formyl; acyl (e.g. C₁₋₇ alkylacyl, C₅₋₂₀ arylacyl); acylhalide; carboxy; ester; acyloxy; amido; acylamido; thioamido; tetrazolyl; amino; nitro; nitroso; azido; cyano; isocyano; cyanato; isocyanato; thiocyano; isothiocyano; sulfhydryl; thioether (e.g., C₁₋₇ alkylthio); sulphonic acid; sulfonate; sulphone; sulfonyloxy; sulfinyloxy; sulfamino; sulfonamino; sulfinamino; sulfamyl; sulfonamido; C₁₋₇ alkyl [including, e.g., unsubstituted C₁₋₇ alkyl, C₁₋₇ haloalkyl, C₁₋₇ hydroxyalkyl, C₁₋₇ carboxyalkyl, C₁₋₇ aminoalkyl, C₅₋₂₀ aryl, C₁₋₇ alkyl); C₃₋₂₀ heterocyclyl; and C₅₋₂₀ aryl (including, e.g., C₅₋₂₀ carboaryl, C₅₋₂₀ heteroaryl, C₁₋₇ alkyl-C₅₋₂₀ aryl and C₅₋₂₀ haloaryl)] groups.

The term “ring structure” as used herein, pertains to a closed ring of from 3 to 10 covalently linked atoms, yet more preferably 3 to 8 covalently linked atoms, yet more preferably 5 to 6 covalently linked atoms. A ring may be an alicyclic ring, or aromatic ring. The term “alicyclic ring,” as used herein, pertains to a ring which is not an aromatic ring.

The term “carbocyclic ring”, as used herein, pertains to a ring wherein all of the ring atoms are carbon atoms.

The term “carboaromatic ring”, as used herein, pertains to an aromatic ring wherein all of the ring atoms are carbon atoms.

The term “heterocyclic ring”, as used herein, pertains to a ring wherein at least one of the ring atoms is a multivalent ring heteroatom, for example, nitrogen, phosphorus, silicon, oxygen or sulphur, though more commonly nitrogen, oxygen, or sulphur. Preferably, the heterocyclic ring has from 1 to 4 heteroatoms.

The above rings may be part of a “multicyclic group”.

Typically, the ratio of amphiphile compound to drug is within the range of 0.1-20:1; a preferred ratio is 1-10:1 and a more preferred ratio is around 5:1 by weight.

Typically, the ratio of amphiphile compound to drug to pharmaceutically acceptable carrier may be about 1-5 mg:1 mg:1 g.

The compositions may be delivered to the human or animal body by a range of delivery routes including, but not limited to: gastrointestinal delivery, including orally and per rectum; parenteral delivery, including injection, patches, creams etc; mucosal delivery, including nasal, inhalation and via pessary. In a preferred embodiment, the compositions are administered via parenteral, oral or topical routes and most preferably orally or by an intravenous route.

In addition to the peptide conjugate and amphiphile as described above, the pharmaceutical compositions may comprise a pharmaceutically acceptable excipient, carrier, diluent, buffer, stabiliser or other materials well known to those skilled in the art. Such materials should be non-toxic and should not interfere with the efficacy of the composition. The precise nature of the carrier or other material may depend on the route of administration, e.g. parenteral, oral or topical routes.

Pharmaceutical compositions for oral administration may be in tablet, capsule, powder or liquid form. A tablet may include a solid carrier such as gelatine or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

When tablets are used for oral administration, typically used carriers include sucrose, lactose, mannitol, maltitol, dextran, corn starch, typical lubricants such as magnesium stearate, preservatives such as paraben, sorbin, anti-oxidants such as ascorbic acid, alpha-tocopherol, cysteine, disintegrators or binders. When administered orally as capsules, effective diluents include lactose and dry corn starch. Liquids for oral use include syrups, suspensions, solutions and emulsions, which may contain a typical inert diluent used in this field, such as water. In addition, the composition may contain sweetening and/or flavouring agents.

For intravenous, cutaneous or subcutaneous injection, or injection at the site of affliction, the composition will be in the form of parenterally acceptable aqueous solution which is pyrogen-free and has suitable pH, isotonicity and stability. Those of relevant skill in the art are well able to prepare suitable solutions using, for example, isotonic vehicles such as sodium chloride for injection. Preservatives, stabilisers, buffers, antioxidants and/or other additives may be included, as required.

A suitable daily dose can be determined based on age, body weight, administration time, administration method, etc. While the daily doses may vary depending on the condition and body weight of the patient, and the nature of the drug, a typical oral dose is about 0.1 mg-2 g/person/day, preferably 0.5-100 mg/person/day.

The invention will now be illustrated by the following Examples, which refer to the following figures:

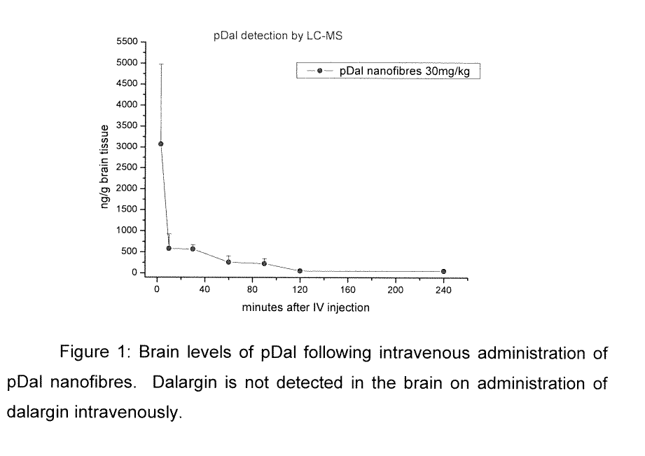

FIG. 1: Brain levels of pDal following intravenous administration of pDal nanofibres. Dalargin is not detected in the brain on administration of dalargin intravenously.

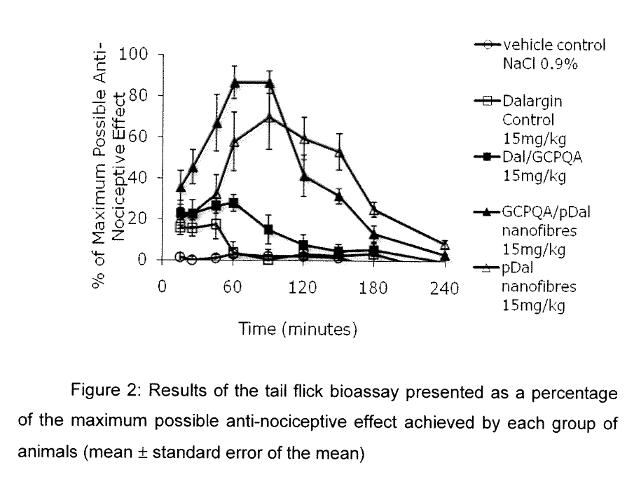

FIG. 2: Results of the tail flick bioassay presented as a percentage the maximum possible anti-nociceptive effect achieved by each group of animals (mean±standard error of the mean)

### EXPERIMENTAL METHODS

### Synthesis of Acetylated Quaternary Ammonium Palmitoyl Glycol Chitosan (GCPQA)

Glycol chitosan (2 g, GC) was degraded in a solution of HCl (152 mL, 4M) for 24 hours, dialysed against deionised water (5 L) in a dialysis bag [12-14 kDa molecular weight cut off (MWCO)] with 6 changes over 24 h. After freeze-drying the polymer (100 mg) was dissolved in sodium bicarbonate solution (0.09M, 10 mL) to which was added absolute ethanol, and reacted with Palmitic acid N-hydroxysuccinamide (792 mg, PNS) ester dissolved in ethanol (150 mL). The reaction solution was left to stir for 72h and protected from light. The ethanol was evaporated off under vacuum and residual aqueous liquid extracted with diethyl ether (3×200 mL). The solution was then dialysed against deionised water (5 L) in a dialysis bag (12-14 kDa MWCO) with 6 changes over 24 h and lyophilized.

Quaternisation of the palmitoyl carbohydrate was achieved by dispersing PGC (300 mg) in N-methyl-2-pyrrolidone (25 mL) and reacting PGC with methyl iodide (1.0 g) at 36° C. under a stream of nitrogen for 3 h in the presence of sodium iodide (45 mg) and sodium hydroxide (40 mg) which were all added dispersed or dissolved in absolute ethanol (4 mL). The product was subsequently precipitated by adding diethyl ether (200 mL). The precipitate was collected, redissolved in water (100 mL]) and dialysed against NaCl (0.1 M, 5 L, 3 changes), followed by deionised water (5 L and 6 changes) before freeze-drying. The quaternary ammonium palmitoyl glycol chitosan (GCPQ) thus obtained (100 mg) was dissolved in sodium bicarbonate (0.08M, 10 mL) and methanol (20 mL). To this solution was added a solution of acetic anhydride (0.012 mL) in methanol (5 mL). The reaction was stirred for 24 h and then stopped by adding NH4OH. The resulting liquid was then dialyzed against deionised water (5 L with 6 changes) and lyophilized.

Synthesis of Palmitoyl Dalargin (pDal)

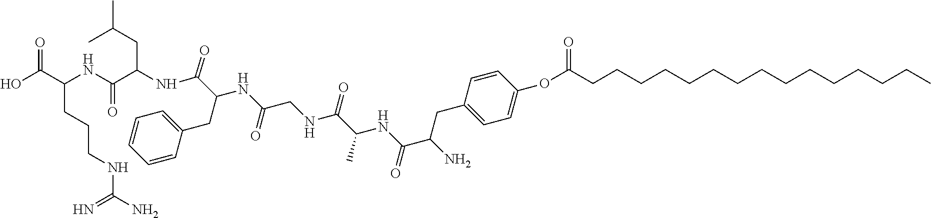 [SMILES: CCCCCCCCCCCCCCCC(=O)Oc1ccc(CC(N)C(=O)N[C@H](C)C(=O)NCC(=O)NC(Cc2ccccc2)C(=O)NC(CC(C)C)C(=O)NC(CCCNC(=N)N)C(=O)O)cc1]

pDal was synthesised by first synthesising dalargin using manual solid-phase synthesis and standard fluorenylmethoxycarbonyl (Fmoc) protected amino acids, followed by conjugation of dalargin to palmitic acid.

To the H-Arg-2-Cl-Trt resin (0.943 g, 0.53 mmoles g⁻¹) was added dimethyl formamide (DMF, 4-8 mL) and the resin left to swell for 1 hour. To swollen resin was then added Fmoc orthogonally protected amino acid (Fmoc-L-Leucine, 0.44 g, 1.25 mmoles), O-(1H-benzotriazole-1-yl)-N,N,N′,N′-tetramethyluronium hexafluorophosphate (HBTU, 0.47 g, 2.5 mmoles) and 1-Hydroxybenzotriazole (HOBt, 0.436 μL, 2.5 mmoles) all dissolved in a minimum volume of dimethyl formamide (DMF) To the reaction was then added N,N-Diisopropylethylamine (DIEA, 191 mg, 1.48 mmoles) and the reaction allowed to proceed for 30 mins. For each amino acid residue coupled, the above procedure was performed twice. After coupling each residue the Kaiser test (16) was performed to ensure coupling had taken place. Deprotection of the Fmoc moiety after washing the resin with DMF (150 mL) was achieved by adding piperidine (20% v/v in DMF, 10 mL) to the resin beads, which was then agitated for 10 minutes (performed twice). The process detailed above was repeated for each amino acid residue until synthesis of the peptide was complete. All peptide synthesis steps were performed at room temperature. Once peptide synthesis had been completed, the resin was washed with copious amounts of DMF (250 mL), followed by copious amounts of dichloromethane (DCM, 100 mL) and then by a mixture of DCM, methanol (1:1, 200 mL). The peptide bound resin was dried under vacuum and then transferred to a pre-weighed glass container and left in a dessicator under vacuum for 24 hours.

Triethylamine (665 μL X mg, 4.8 mmol) was added to a dispersion of the peptide bound to the resin (0.266 g, 0.1 mmol) preswelled in DMF (8 mL) and to the resultant suspension was added dropwise the N-hydroxysuccinimide ester of palmitic acid (282 mg, 0.85 mmol) in DMF (8 mL). The reaction was left for 24 h at 25° C., during which time the suspension was agitated (120 rpm). The mixture was then concentrated in vacuo to remove volatile products and the residue dispersed in DMF (4 mL). The DMF suspension was filtered and the residue washed with copious amounts of DMF (100 mL). The product bound to the resin was treated with piperidine in DMF (20% v/v, 20 mL) for 20-25 minutes. After washing with DMF and filtration, cleavage of the peptide chain from the resin was performed by treatment with the reagent R (trifluoroacetic acid, ethanediol, thioanisole, anisole—90:3:5; 2, 1 mL for each 0.1 mg of the resin). The reaction mixture was evaporated under vacuum, the peptide precipitated with cold purified water (4° C. 4 mL) and the precipitate collected by centrifugation (5,000 rpm×30 minutes and repeated twice, Z323 Hermle centrifuge, VWR, Poole, UK). The pellet was then redissolved in acetonitrile and freeze dried.

Peptide purification was achieved using semi-preparative reverse-phase HPLC (RP-HPLC). Crude peptide (6-8 mg ml⁻¹) dissolved in dimethylsulfoxide (DMSO) and mobile phase was chromatographed over a semi-preparative Waters Spherisorb ODS₂ C18 column (10 mm×250 mm, pore size=10 μm) using a 30 minute gradient from 5% aqueous (solvent A) —100% organic (solvent B) and a flow rate of 6 mL/min (solvent A (TFA—0.02% v/v) and solvent B (acetonitrile, water—90:10 TFA 0.016%). Peptides were detected at 230 nm using a Waters 486 variable wavelength UV detector. Fractions containing the peptide were pulled together and freeze-dried.

### Mass Spectrometry (MS)

Low resolution nominal mass measurement were done using ThermoQuest Navigator from Thermo Finnigan (now Thermo Electron/Thermo Fisher Scientific) operated under Electrospray ionization (ESI) and atmospheric pressure chemical ionization (APCI) interfaces for liquid sample introduction.

Samples were prepared in 50:50 acetonitrile: water+0.1% formic acid.

Nuclear Magnetic Resonance (NMR) ¹H NMR and ¹H—¹H COSY experiments were performed on pDal (dissolved in DMSO) on a Bruker AMX 400 MHz spectrometer (Bruker Instruments, Coventry, UK). Analyses were performed at a temperature of 45-50° C.

### Horizontal Attenuated Total Reflectance Fourier-Transformed Infrared Spectroscopy (HATR-FTIR)

The infrared absorption spectra for pDal was recorded using a Perkin Elmer Spectrum 100 FTIR Spectrometer equipped with a Universal Attenuated Total Reflectance accessory and a zinc selenide crystal (4000-650 cm⁻¹) and Spectrum FTIR software. A background spectrum was recorded on a clean zinc selenide window before a sample spectrum was recorded.

Preparation of Self-Assembling pDal Nanofibres

Self assembled pDal nanofibres were prepared by vortexing a suspension of pDal (1 mg mL⁻¹) in water, followed by probe sonication (MSE soniprep 150, MSE London, UK) with the instrument set at 50% of its maximum output for 20 minutes on ice. Self-assembled pDal nanofibres were also prepared by applying a short microwave burst (Microwave Panasonic NN-3454 800W-D, Panasonic UK, Bracknell, Berks) for 10 seconds with the power level at Simmer (240 W) and/or High (800 W).

The nanofibres were imaged using transmission electron microscopy (TEM). A drop of sample liquid was placed on Formvar©/Carbon Coated Grid (F196/100 3.05 mm, Mesh 300, Tab Labs Ltd, England). Excess sample was blotted off with No. 1 Whatman Filter paper and negatively stained with uranyl acetate (1% w/v). Imaging was carried out using an FEI CM120 BioTwin Transmission Electron Microscope (Philips, XYZ town, XYZ country). Digital Images were captured using an AMT digital camera.

Intravenous Administration of pDal Nanofibres

ICR (CD-1) male out bred mice (18-24 g, 4 weeks old, Harlan, Oxon, UK) were used for the pharmacokinetics evaluations while ICR (CD-1) male out bred mice (22-28 g, 4-5 weeks old) were used for the pharmacodynamics evaluations. The animals were housed in groups of 5 in plastic cages in controlled laboratory conditions with ambient temperature and humidity maintained at ˜22° C. and 60% respectively with a 12-hour light and dark cycle (lights on at 7:00 and off at 19:00). Food and water were available ad libitum and the animals acclimatised for 5-7 days prior to any experiments in the Animal House, School of Pharmacy, University of London (London, UK). Animals were only used once and were acclimatised in the testing environment for at least 1 hour prior to testing. All experiments were performed in accordance with the recommendations and policies of the Home Office (Animals Scientific Procedures Act 1986, UK) and the Ethics Committee of the School of Pharmacy, University of London guidelines for the care and use of laboratory animals.

### Pharmacokinetics Studies

Groups (n=5) of animals were administered either: NaCl (0.9% w/v), Dalargin, Dalargin-GCPQA, pDal and pDal-GCPQA. Animals received a dalargin dose of 30 mg kg⁻¹ and sodium chloride was used as the disperse phase. The volume of injection was 0.2 mL per 25 g of mouse weight. At various time points, animals were killed and their brain, liver and plasma analysed.

### UPLC-MS/MS Analysis of Biological Matrices

Blood samples (0.4-0.8 mls per mouse) were collected into a chilled syringe and transferred into evacuated sterile spray coated (with tripotassium ethylenediamine tetraacetic acid −3.6 mg) medical grade PET tubes (3×75 mm K3E Vacutainer©, BD Biosciences, UK) and maintained on ice (4° C.) until centrifugation. There is no dilution effect in spray coated tubes. Plasma was obtained as the supernatant after centrifugation of blood samples at 1,600g or 4800 rpm for 15 minutes at 4° C. with a Z323 Hermle centrifuge, VWR, Poole, UK) and was pipetted into 1.5 mL centrifuge tubes and stored at −80° C. for later use.

Brain and Liver were immediately frozen in liquid Nitrogen after being taken from the mouse. On the day of analysis all plasma, brain and, liver samples were removed from the freezer and thawed. The brain and liver weights were determined and 2 mL water per g of brain was added to each (equivalent to 2 g of solvent to 1 g of brain). All brain and liver samples were homogenised using the Tomtec Autogeizer (cutter). The plasma samples, once thawed, were sub-aliquoted (50 uL) into 1.5 mL Matrix tubes. The brain and liver samples, once homogenised, were sub-aliquoted (100 uL) into 1.5 mL Matrix tubes. Analyses were carried out on a Mass Spec Instrument (Applied Biosystems API4000, Mode of operation: Positive-ion/Turbo Ionspray, Source Temperature: 625° C., Software version: Analyst 1.4.2, Multiple Reaction Monitoring Transitions for Dalargin: 726.6->136.2, Palmitoyl Dalargin 964.8->136.2, [D-Ala2]-Leucine 570.4->136.1, Pump Instrument Type: JASCO XLC, HPLC Column (type/size): Thermo Gold (Aqua) 30×3 mm, pore size=3 μm, Column temp (° C.)=50° C., Flow rate=1.0 mL min⁻¹, Volume split from LC into source: No split, Run time=2.5 min, Injection volume=20 μL, Solvent A: 10 mM Ammonium acetate, Solvent B: Methanol, Autosampler Instrument Type: Presearch PAL CTC Autosampler.

Gradient elution: (if applicable)

| Time | Solvent B | Flow Rate |
|---|---|---|
| (min) | (%) | (mL/min) |
| 0 | 20 | 1.0 |
| 0.8 | 90 | 1.0 |
| 1.8 | 90 | 1.0 |
| 1.81 | 20 | 1.0 |

### Extraction Procedure

The extraction volume was 250 μL, the internal standard concentration was 10 ng mL⁻¹. Ethanol (50 μL) was added to all samples. Appropriate extraction volume of working “IS” solution added to all standards and samples. Samples were shaken for 20 mins on a vortex mixer then centrifuged for 15 mins at 2,465 g and the supernatant injected.

### Phamacodynamics Studies

Groups (n=6) of animals were administered either: NaCl (0.9% w/v), Dalargin, Dalargin-GCPQA, pDal and pDal-GCPQA. Animals received a dalargin dose of 15 mg kg-1 and sodium chloride was used as the disperse phase. The volume of injection was 0.2 mL per 25 g of mouse weight.

Anti-nociception was assessed in mice using the tail flick warm water bioassay (17, 18). The protruding distal half of the tail (4-5 cm) of confined mice in a Plexiglas restrainer was immersed in circulating warm water maintained at 55° C.±0.1° C. (19, 20) by a thermostatically controlled water bath (W14, Grant Instruments, Cambridge Ltd, Herts, UK). Before any experiment was performed the temperature was checked using a thermometer (Gallenkamp, Griffin, THL-333-020L, 76 mm×1 mm, UK). The response latency times, in centiseconds, recorded for each mouse to withdraw its tail by a “sharp flick” were recorded using a digital stopwatch capable of measuring 1/100th of a second. The first sign of a rapid tail flick was taken as the behavioural endpoint which followed in most cases 1-3 slow tail movements.

Two separate withdrawal latency determinations (separated by ≧20 sec) were averaged. The tails of the mice were wiped dry immediately after testing to prevent the hot water from clinging to the tail producing erythema. Mice not responding within 5 sec were excluded from further testing (Baseline cut-off=5 seconds) and the baseline latency was measured for all mice 2 hours prior testing. Maximum possible cut-off was set to 10 seconds to avoid unnecessary damage to the tail (19). A maximum score was assigned (100%) to animals not responding within 10 seconds to the thermal stimuli. The response times were then converted to percentage of maximum possible effect (% MPE) by a method reported previously (20). Briefly, percent antinociception was calculated as 100%×(test latency-baseline latency)/(10 seconds-baseline latency). Data are presented as the mean±SEM for groups of 6 mice per group. An analgesic responder was defined as one whose response tail flick latency was two or more times the value of the baseline latency (21).

### Results and Discussion

Palmitoyl dalargin (pDal), a derivative of the opioid analgesic peptide Dalargin has been synthesized by attachment of a palmitic tail to the side chain of the last amino acid in the sequence. This lipid tail enable the molecules of pDal to self assemble into nanofibres. Morphological investigations have shown that the high axial ratio micellar aggregates can form either cylindrical or twisted nanofibres.

After intravenous administration, pDal is detected in the brain. Dalargin is not detected in the brain after the intravenous administration of dalargin formulations (FIG. 1)

Analgesia was defined as a tail flick latency for an individual animal that was twice its baseline latency or more. The Maximum Possible Effect was calculated as

% MPE=[(post drug latency-predrug latency)/(cut off time-predrug latency)]×100

The results show that an increased antinociceptive effect was obtained with the formulations containing the GCPQA and with only the animals dosed with pDal/GCPQA was the Maximum Possible Effect obtained. Dalargin alone is unable to exert an antinociceptive effect when administered intravenously (FIG. 2).

### REFERENCES

1. Chew S. Y., Park T. G. Nanofibres in regenerative medicine and drug delivery. Advanced Drug Delivery Reviews. 61 (2009) 987

2. Cui H., Webber M. J., Stupp S. I. Self-assembly of peptide amphiphiles: From molecules to nanostructures to biomaterials. Biopolymers Peptide (2010) 94:1 1-18

3. Kalenikova, E. I., Dmitrieva O. F., Korobov, N. N., Zhukova, S. V.

1. A composition comprising nanofibres for the delivery of a peptide across the blood brain barrier in a method of therapy of the human or animal body, wherein the nanofibres comprise a peptide conjugated to a lipophilic group.
wherein the lipophilic group is enzymatically cleavable from the peptide; and
wherein the peptide is a neuroactive agent.

2. (canceled)

3. A composition according to claim 1, wherein the lipophilic group comprises a C₄-₃₀ alkyl group, a C₄-₃₀ acyl group, a C₄-₃₀ alkenyl group, a C₄-30 alkynyl group, a C₅-₂o aryl group, a multicyclic hydrophobic group with more than one C₄-C₈ ring structure, a multicyclic hydrophobic group with more than one C₄-C₈ heteroatom ring structure, a polyoxa C₁-C₄ alkylene group, a poly(lactic acid) group, a poly(lactide-co-glycolide) group or a poly(glycolic acid) group.

4. A composition according to claim 3, wherein the lipophilic group has the general formula —C(═O)R¹ wherein R¹ is C₄-₂₀ alkyl.

5. (canceled)

6. A composition according to claim 1, wherein the peptide is dalargin.

7. A composition according to claim 1, wherein the composition further comprises an amphiphile compound which is preferably selected from sorbitan esters, polysorbate esters, poly(ethylene glycol)ethers, poly(ethylene glycol)esters, poly(ethylene glycol)-poly(propylene glycol) block copolymers, phospholipids, chitosans, dextrans, alginic acids, starches, guar gums and their derivatives.

8. A composition according to claim 7, wherein the amphiphile compound is acetylated quarternary palmitoyl glycol chitosan (GCPQA).

9. A composition according to claim 1 further comprising a pharmaceutically acceptable carrier.

10. A composition according to claim 1, which is orally or intravenously administered to a human or animal body.

11. A method of medical treatment wherein a composition according to claim 1 is administered to a human or animal body.

12. A method according to claim 11, which is the treatment of schizophrenia, obesity, pain, a sleep disorder, a psychiatric disease, a neurodegenerative condition, brain cancer, or an infective diseases.

13. A method of forming a composition according to claim 1 comprising probe sonicating an aqueous dispersive of a peptide conjugated to a lipophilic group.

14. A method of forming a composition according to claim 1 comprising conjugating a lipophilic group to a peptide and forming nanofibres from the conjugate.

15. A compound comprising Dalargin which has a lipophilic group conjugated to an aminoacyl group.

16. A compound according to claim 15 in which the lipophilic group is a C₆₋₂₄ acyl group.

17. A composition comprising the compound of claim 15 and a carrier or diluent, preferably a pharmaceutical composition wherein the carrier or diluent is pharmaceutically acceptable.

18. A method of synthesising the compound of claim 15 by conjugating the corresponding lipophilic carboxylic acid or activated derivative, to the side chain of the terminal amino acid group.

19. The compound according to claim 15, wherein the lipophilic group is conjugated to the aminoacyl group via a side chain or a linker.

20. The compound according to claim 16, wherein the lipophilic group is a C₁₆₋₂₂ group.
